(19) United States
(12) Patent Application Publication
(10) Pub. No.: US 2005/0154035 A1
(43) Pub. Date: Jul. 14, 2005

(54) Methods of using zonisamide as an adjunctive therapy for partial seizures

(75) Inventor: Ivan Lieberburg, Berkeley, CA (US)

Correspondence Address: Finnegan, Henderson, Farabow,;Garrett & Dunner, L.L.P., 1300 I Street, N.W., Washington, DC, 20005-3315, US

(73) Assignee: Eisai Co., Ltd., Tokyo (JP)

(21) Appl. No.: 10/753,955

(22) Filed: Jan. 9, 2004

(51) Int. Cl. A61K 31/42

(52) U.S. Cl. 514/379

(57) ABSTRACT

Methods of using zonisamide as an adjunctive therapy for partial seizures are disclosed. In particular, the methods enhance the safety of patients taking pharmaceutical formulations of zonisamide by providing information that increases the awareness of rhabdomyolysis and/or elevated CPK as possible side effects; wherein the patients and/or prescribing physicians and other medical care providers are advised to monitor for such conditions and employ methods that will improve the therapeutic outcome in the few patients who experience rhabdomyolysis and/or elevated CPK associated with zonisamide therapy.

### FIELD OF THE INVENTION

The present invention generally relates to methods of using zonisamide (3-benzisoxazole methylene sulfonamide) as an adjunctive therapy for partial seizures.

### BACKGROUND OF THE INVENTION

In the United States, over 2 million serious adverse drug reactions (ADRs) occur ever year, with 100,000 associated deaths. This places ADRs as the fourth leading cause of death, ranking ahead of pulmonary disease, diabetes, AIDS, pneumonia, accidents, and automobile deaths. Compounding this problem is the fact that ADRs increase exponentially in patients who take four or more medications concurrently. (See http://www.fda.gov/cder/drug/drugReactions/default.htm, last checked Aug. 20, 2003.)

Most drugs are approved by a Food and Drug Administration review process after an average of 1,500 patient exposures. Clinical trials involving this number of subjects (both healthy volunteers and patients in need of the therapeutic effect of the drug under review) provide a statistically relevant sample of the population from which an assessment of safety and efficacy can be evaluated. However, some drugs have very rare toxicity profiles. Bromfenac, for example, causes hepatotoxicity in 1 out of 20,000 patients. For drugs with rare toxicity, more than 100,000 patients must be exposed to generate a warning signal for the adverse event. In instances where an adverse event is identified in association with a human therapeutic, government regulations require a post-approval follow-up after the drug has been taken to market.

Examples of very serious post-marketing events that have been identified in the recent past include Fen-Phen (fenfluramine-phentermine combination therapy) for weight loss and Rezulin (troglitazone) for diabetes, both of which were later removed from the market because the ADR risks outweighed the therapeutic benefits. Statistical and clinical analysis of large adverse event databases collected by post-marketing surveillance is one method by which identification of the rarer ADRs can be made. For more background on the occurrence and identification of ADRs see, for example, Lazarou, J. et al., JAMA 279(15): 1200-1205 (1998), and Gurwitz, J. H. et al. Am J. Med. 109(2): 87-94 (2000). For a discussion of techniques and difficulties inherent in identifying ADRs in adjunctive therapies of epileptic seizures, see French, J. Epilepsia 43(9): 951-955 (2002), which is hereby incorporated by reference in its entirety.

While Rezulin and Fen-Phen are notable for their extreme and potentially irreversible nature, other adverse drug reactions can be minimized or more easily reversed if they are recognized early, and appropriate and timely medical intervention is made. A few examples of frequently reversible adverse events are cardiac arrhythmias, liver function abnormalities, and irregularities in hematopoiesis. Thus, there remains a need for methods for identifying, for detecting and for treating adverse events associated with drug therapy, in a timely and informed manner.

### DESCRIPTION OF THE INVENTION

Unexpectedly, it has been found that zonisamide therapy in a very small percentage of patients worldwide can precipitate rhabdomyolysis and/or serum CPK elevation (about 1:244,491, based on combining the reported cases of rhabdomyolysis and elevated CPK in both the U.S. and Japan). It also has been found that that by curtailing (either by removal, reduction, or tapering off) the administration of zonisamide dosing, alone or in conjunction with other concomitant medications, alleviation and minimization of this severe adverse event is possible. This is particularly the case when medical intervention to manage the disease and/or removal, reduction, or tapering off of zonisamide is instituted rapidly.

Accordingly, the present invention is directed to methods of using zonisamide for a regulatory agency approved use (e.g., as an adjunctive therapy for partial seizures). The methods improve the safety of zonisamide therapy for patients receiving administrations of the drug, or those who are in need of zonisamide therapy.

In some embodiments, the methods of using zonisamide as an adjunctive therapy for partial seizures improves the safety and health of patients FINNEGAN HENDERSON taking zonisamide by increasing the awareness of the patient or patient's guardian that rhabdomyolysis and/or creatine phosphokinase (CPK) elevation are possible side effects. Accordingly, a patient may be provided with a therapeutically effective amount of zonisamide, and the patient or the patient's guardian may be informed that muscle stiffness, muscle pain, muscle weakness, fever, discolored urine or altered consciousness are symptoms of rhabdomyolysis and/or creatine phosphokinase (CPK) elevation that require prompt medical evaluation if such symptoms are experienced by the patient. As a result, the patient or patient's guardian can monitor for signs and symptoms of rhabdomyolysis and/or creatine phosphokinase (CPK) elevation, and seek medical attention if such symptoms occur in order to obtain appropriate tests, diagnosis, and treatment. In some embodiments, the present methods reduce the risk of rhabdomyolysis and/or creatine phosphokinase (CPK) elevation in patients receiving zonisamide therapy.

In other embodiments, the present invention provides methods of using zonisamide as an adjunctive therapy for partial seizures comprising informing a prescribing physician or other medical professional (e.g., an emergency medical worker) that rhabdomyolysis and/or creatine phosphokinase (CPK) elevation may result from zonisamide therapy and to monitor a patient who is prescribed zonisamide as an adjunctive therapy for partial seizures for muscle stiffness, muscle pain, muscle weakness, fever, discolored urine or altered consciousness. The prescribing physician or other medical professional also may be advised that when muscle stiffness, muscle pain, muscle weakness, fever, discolored urine or altered consciousness is observed, an appropriate diagnostic be employed to determine whether rhabdomyolysis and/or creatine phosphokinase (CPK) elevation is present. In addition, the prescribing physician or other medical professional may be advised to remove, reduce, or taper off the zonisamide dosing in the patient, and initiate appropriate supportive therapy for the underlying condition(s). In this manner, the present methods enable prescribing physicians and other health care professionals to recognize and minimize the risk associated with an adverse event, namely rhabdomyolysis and/or creatine phosphokinase (CPK) elevation, which may occur in some patients who receive zonisamide therapy.

The present methods also include methods of administrating zonisamide as an adjunctive therapy for partial seizures comprising providing packaging that includes a pharmaceutical formulation of zonisamide along with information providing a warning that zonisamide may cause rhabdomyolysis and/or creatine phosphokinase (CPK) elevation in some patients and that muscle stiffness, muscle pain, muscle weakness, fever, discolored urine and altered consciousness are symptoms of rhabdomyolysis and/or creatine phosphokinase (CPK) elevation; and providing the packaging to a patient who has been prescribed zonisamide.

The medical information provided in any of the above described methods concerning the signs and symptoms of rhabdomyolysis may alternatively be provided in layman's terms, so as to be better understood by patients or non-medical professionals. Those of skill in the medical art are familiar with the various layman's terms that can be used to describe the symptoms of rhabdomyolysis.

Other advantages and uses of the present invention will become apparent to those skilled in the art in studying this disclosure; therefore this recitation is not intended to limit the scope of the claims attached hereto.

### DESCRIPTION OF THE EMBODIMENTS

Zonisamide is an anti-seizure drug, chemically classified as a sulfonamide and unrelated to other anti-seizure agents. Antiepileptic drugs are commonly abbreviated as “AEDs”. The active ingredient is zonisamide, 1,2-benzisoxazole-3-methanesulfonamide. Zonisamide was approved in 2000 for the adjunctive treatment (i.e., taken in conjunction with one or more other AEDs) treatment of epilepsy in the United States, while it was first introduced in Japan approximately 12 years ago, where it also has been used as monotherapy, i.e., without other AEDs as concomitant therapeutics. Zonisamide is not known to be a hepatic enzyme inducer and has been administered adjunctively with almost all of the other regulatory-approved AEDs either in the United States or abroad.

The precise mechanism(s) by which zonisamide exerts its anti-seizure effect is unknown. Zonisamide may produce anti-seizure effects through action at sodium and calcium channels. In vitro pharmacological studies suggest that zonisamide blocks sodium channels and reduces voltage-dependent, transient inward currents (T-type Ca²⁺ currents), consequently stabilizing neuronal membranes and suppressing neuronal hypersynchronization, thus suppressing hyperexcitablity in epileptic foci. In vitro binding studies have demonstrated that zonisamide binds to the GABA/benzodiazepine receptor ionophore complex in an HENDERSON allosteric fashion, which does not produce changes in chloride flux. Other in vitro studies have demonstrated that zonisamide (10-30 μg/mL) suppresses synaptically-driven electrical activity without affecting postsynaptic GABA or glutamate responses (cultured mouse spinal cord neurons) or neuronal or glial uptake of [³H]-GABA (rat hippocampal slices). Thus, zonisamide does not appear to potentiate the synaptic activity of GABA. In vivo microdialysis studies demonstrated that zonisamide facilitates both dopaminergic and serotonergic neurotransmission. Zonisamide also has weak carbonic anhydrase inhibiting activity (about 1/50^{th} the inhibition compared to acetazolamide), and this pharmacologic effect is not thought to be a major contributing factor in the anti-seizure activity of zonisamide.

Zonegran® (the human therapeutic pharmaceutical formulation containing zonisamide) is indicated as adjunctive therapy for the treatment of partial seizures in adults and is supplied by prescription in the form of 25, 50, and 100 mg capsules. The capsules may be divided, so as to offer smaller increments in dosage. Recommended dosing is once or twice daily, the recommended daily dose of 100 mg at the initiation of therapy should not be divided. Zonegran® is given orally and can be taken with or without food. While other therapeutic uses of zonisamide have been reported, such as treatment of obesity and eating disorders, treatment of neuropathic pain, prophylaxis of migraine attacks, and treatment of mania, these are not indications approved by the Food and Drug Administration (FDA) in the United States, and so are called “off-label” uses. Off-label uses, which are within the discretion of the prescribing physician to write, are also encompassed in the methods presented herein.

Prescribing physicians are informed in the product insert (which contains prescribing information approved by the FDA) that, because of the long half-life of zonisamide, up to two weeks may be required to achieve steady state levels upon reaching a stable dose or following dosage adjustment. Although the regimen described below has been shown to be tolerated, the prescriber may wish to prolong the duration of treatment at the lower doses in order to fully assess the effects of zonisamide at steady state, noting that many of the side effects of zonisamide are more frequent at doses of 300 mg per day and above. Although there is some evidence of greater response at doses above 100-200 mg/day, the increase appears small and formal dose-response studies have not been conducted.

The initial dose should be 100 mg daily. After two weeks, the dose may be increased to 200 mg/day for at least two weeks. It can be increased to 300 mg/day and 400 mg/day, with the dose stable for at least two weeks to achieve steady state at each level. Evidence from controlled trials suggests that Zonegran® doses of 100-600 mg/day are effective, but there is no suggestion of increasing response above 400 mg/day.

Adjunctive therapy for partial seizures in adults denotes that these patients are already on other anti-epileptic medications, but that they are continuing to seize at a rate that has been deemed by their treating physician to require additional (add-on) therapy. For a recent review of AEDs currently available to American physicians, their efficacies for particular types of epileptic seizures and associated ADRs, see: Ilo Leppik, Epilepsia 42(Suppl.4): 1-6 (2001).

The use of multiple anti-epileptic medications in the adjunctive setting increases the likelihood of confluent or interactive ADRs, and also may confuse the treating physician as to the causal agent. For instance, when an attending medical professional is presented with a patient taking a combination of medications and manifesting a particular side-effect, it is difficult to diagnose which of the patient's medications (or combination of medications) is responsible for the observed side effect. Typically, the attending physician must consult the medical literature of known adverse events to identify drug(s) that are most likely to cause the observed side-effects. Known adverse events may also be found in the package drug inserts for each drug. The drug(s) having the higher likelihood of causing the observed side-effects are usually reduced or withdrawn first. When such options are exhausted, the patient may have to be systematically withdrawn from the various drugs until the cause is identified. Since zonisamide is typically prescribed as an adjunctive therapy, it presents such complications when side-effects occur.

This situation is further complicated when side-effects occur that are not normally associated with a particular drug. For example, zonisamide was not previously known to be linked with rhabdomyolysis in patients receiving ZONEGRAN® therapy. Given this absence of knowledge concerning the incidence of such adverse events, a medical professional would not suspect zonisamide to be the likely agent responsible for causing rhabdomyolysis in a patient exhibiting the relevant symptoms. Consequently, the attending medical professional would have no obvious reason to withdraw such a patient from zonisamide, and would allow the therapy to continue while searching for other causes of the rhabdomyolysis. However, a careful review of the data generated in American clinical trials, as well as in ADR reports gathered once commercial marketing began, has yielded the discovery that zonisamide may independently induce rhabdomyolysis in a small number of patients, and has implicated rhabdomyolysis in patients receiving zonisamide as an adjunctive therapy. Accordingly, the present invention is directed to methods of increasing the safety of zonisamide therapy in view of its newly discovered role in rhabdomyolysis.

Rhabdomyolysis is a condition caused by skeletal muscle injury and release of muscle cell contents into the circulation. Many insults can precipitate rhabdomyolysis and myoglobinuria (the filtration of myoglobin from injured muscle into the urine). Disruption of the muscle cell membrane may result from a direct mechanical or toxic insult to the membrane, or an inability to maintain ionic gradients across the membrane (as in ischemia, muscle exhaustion or seizures, particularly status epilepticus and clonic seizure). Toxic insult can come from a number of chemical sources including ethanol, pharmaceuticals and illicit drugs. Aldolase and isozymes of creatine phosphokinase (CPK) are enzymes that are relatively specific to striated muscle tissue (vide infra). One or both of these enzymes will usually be found in the serum of a patient who has recently or is undergoing muscle destruction from rhabdomyolysis. Drugs that are known to induce CPK elevation in some small percentage of the population are: alcohol, opiates, cocaine, amphetamine, phencyclidine, barbiturates, cyclosporine, neuroleptics, clofibrate, benzfibrate, lovastatin, antibiotics, amphotericin B, epsilon aminocaproic acid, and some antihistamines.

In some patients, such as those with crush injury, muscle injury is obvious; in others, such as in drug overdose, it may never be apparent. It may occur in the setting of patients with altered mental status, and even in those conscious patients it may occur with minimal symptoms or physical findings. Therefore diagnosis requires a high level of suspicion and appropriate sensitivity to abnormal laboratory values. Gabow P A, Kaehny W D and Kelleher S P The spectrum of rhabdomyolysis. Medicine 1982; 3: 141-152.

Pathogenesis:

Although the causes of rhabdomyolysis are diverse, the pathogenesis appears to follow a final common pathway, ultimately leading to muscle necrosis and release of muscle components into the circulation. This results in an increased cellular permeability to sodium ions by either 1) plasma membrane disruption or 2) reduced cellular energy (ATP) production. Accumulation of sodium in the cytoplasm leads to increased intracellular calcium concentration. This accumulation of calcium is the result of both direct injury to the cell and to increased activity of an Na+/Ca2+ exchanger protein that brings yet more calcium into the cell as it attempts to remove the excess sodium. Depletion of ATP also contributes directly to calcium accumulation since this causes reduction of Ca2+ ATPase activity, which results in less pumping of calcium out of the cell where it is sequestered in the sarcoplasmic reticulum. (See the review by Poels, P. J. E. and Gabreëls, F. J. M. (1993) Rhabdomyolysis: a review of the literature. Clin Neurol & Neurosurg 95: 175-192).

Therefore, the common pathogenic feature of all disease processes causing rhabdomyolysis is an acute rise in the cytosolic and mitochondrial calcium concentration in affected muscle cells that sets off a chain of events ultimately resulting in muscle cell necrosis. Included in the cascade is activation of degradative enzymes such as phospholipase A2 (PLA) and neutral proteases, leading to membrane phospholipid and myofibril damage. Depletion of ATP and mitochondrial damage may be the primary event that sets off this cascade (as with hereditary causes and exertional rhabdomyolysis) or it may occur secondary to the rise in calcium concentration. Either way, mitochondrial damage and depletion of ATP contributes to the pathogenesis via the following: (1) failure of Ca2+ ATPase leading to failure of calcium sequestration and reduced efflux of calcium from the cell; (2) failure of Na+/K+ ATPase leading to increased intracellular sodium and increased Na+-Ca2+ exchange, further contributing to the increased intracellular calcium; and (3) generation of toxic oxygen free radicals such as superoxide, causing further cellular damage.

The combination of these processes is a self-sustaining cycle of events that results in muscle cell lysis and release of intracellular components into the extracellular fluid and systemic circulation. Locally, accumulation of these products in the necrotic tissue may result in microvascular damage, capillary leak and increased compartmental pressures, accompanied by reduced tissue perfusion and ischemia. This combination of factors then potentiates further muscle damage.

Rhabdomyolysis and myoglobinuria pose challenges to physicians in many specialties and to the intensive care doctor in particular, since it may require intensive care for its life threatening complications. Hypovolemia (to the point of shock) may be profound and acute renal failure (ARF) is a common and dangerous complication. Secondarily hyperkalemia and other ionic imbalances, including ion gap acidosis may require electrocardiographic monitoring and emergency dialysis.

Clinical Presentation and Evaluation:

Patients present to an evaluating physician with quite variable symptoms. In the awake, cooperative patient, these may include description of cramping pain in the involved muscle group(s), frequently the calves and lower back; progressive muscle weakness; fever and discoloration of the urine. However, these complaints may be absent 50% of the time, even in an alert patient. Sometimes fever (hyperthermia) or volume depletion are detectable, and the muscles involved may demonstrate stiffness, swelling, tenderness, and a firm consistency. Hemorrhagic discoloration of overlying skin is sometimes evident. However, these findings are not universal, with only about 5% of patients having objective findings of muscle injury on examination. Vanholder et al., Rhabdomyolyis. J Am Soc Nephrology. Vol. 11: 1553-1561. 2000.

Rhabdomyolysis sometimes results in myoglobinuria, the filtration of myoglobin into the urine. In normal skeletal muscle myoglobin content is about 0.3% of the muscle's net weight of and it is released along with other cellular contents after muscle injury and necrosis. Myoglobin is a red respiratory heme pigment closely resembling hemoglobin. The molecular weight of myoglobin is 17,800, approximately one-fourth that of hemoglobin (molecular weight-68,800). Hemoglobin and myoglobin differ in their P-50 value, which is a measure of the oxygen tension of blood. The P-50 for hemoglobin is 26 mm Hg and of myoglobin is 3 mm Hg. The low P-50 of myoglobin correlates with its ability to release oxygen at the low level of oxygen concentration present in the blood during aerobic exertion, thus providing delivery of oxygen to mitochondria to support production of ATP in muscle cells during exertion.

Under normal circumstances, myoglobin concentration ranges from 0 to 0.003 mg/dL in plasma. Fifty percent of plasma myoglobin is bound to a2 globulin at myoglobin concentrations of less than approximately 23 mg/dL. The renal threshold for myoglobin is 0.5 to 1.5 mg/dL. However, the urine level of myoglobin must exceed 100 mg/dL before the urine becomes discolored by myoglobin. The variables that determine if myoglobinuria is visible or otherwise detectable are (1) the plasma level of myoglobin; (2) the extent of the plasma protein binding of myoglobin; (3) the glomerular filtration rate; and (4) the urine flow rate. Serum myoglobin rises prior to elevation of serum creatine phosphokinase (CPK, also referred to as serum creatine kinase or CK). The CPK-MM isoenzyme normally comprises almost all the total CPK enzyme activity in healthy people. When this FINNEGAN HENDERSON particular isoenzyme is elevated, it usually indicates injury or stress to the skeletal muscle. While the serum concentration of myoglobin begins to rise within hours of onset of injury, it returns to normal one to six hours after cessation of injury owing to rapid renal excretion and metabolism to bilirubin, while CPK persists in the blood for days. Since serum concentrations of myoglobin rarely exceed 25 mg/L, urine discoloration is unusual in rhabdomyolysis and is more often taken to suggest hemolysis. Given the limitations to visual detection of myoglobinuria, its use as a diagnostic is not as reliable as measurement of CPK. In a healthy adult, the CPK level in the blood serum varies with a number of factors (gender, race and activity), but normal range is 22 to 198 U/L (units per liter). The primary diagnostic indicator of rhabdomyolysis is an elevated serum creatine phosphokinase (CK) to at least five times the normal value, although it can be elevated to much higher levels. This elevation is generally to such a degree that myocardial infarction and other causes of a raised CK are excluded. Additionally, the CK-MM isoenzyme predominates in rhabdomyolysis, comprising at least 98% of the total value.

Results of laboratory tests on serum samples from an afflicted patient may be notable for several abnormalities. Disruption of the muscle cell membranes releases potassium, phosphate, proteins and purines into the blood stream: hyperkalemia, hyperphosphatemia and hyperuricemia therefore may appear prominently in laboratory values. The hallmark of muscle damage is elevation of creatine phosphokinase (CPK) concentration in the blood, which is present in all patients with rhabdomyolysis. Myocardial infarction and cerebrovascular accident are excluded, as they do not match the severe degree of CPK elevation present in rhabdomyolysis. If necessary, additional information can be gleaned by an isozymic analysis of CPK in the serum. The MB isozyme of CPK is relatively specific to the myocardium and the BB isozyme is relatively specific to the brain. Serum analysis for myoglobin is diagnostic, even when it is not visible in the urine, but this type of detection requires special techniques. Aldolase (aldehyde-lyase), LDH (lactate dehydrogenase) and SGOT and SGPT are also frequently elevated in the serum, but are not dispositive diagnositics since these findings appear in a number of other conditions. Of the three, only aldolase is specific for muscle injury. (However the laboratory test for aldolase is a more expensive test, in large part because this testing has utility limited to detecting rhabdomyolysis since this enzyme is so specific to muscle tissue, but since CPKs are routinely run and isozymes fractionated for myocardial infarctions, using CPK-MM as a diagnostic for rhabdomyolysis has become the standard). SGOT is serum glutamic oxaloacetic transaminase [also called aspartate aminotransferase (AST)], an enzyme present in all tissue, primarily in the liver, heart, and skeletal muscles. It is released into the bloodstream following cell death or injury. Elevated blood levels of SGOT may signal liver, heart, or skeletal muscle disease. The normal range of values for AST (SGOT) is from 5 to 40 units per liter of serum. SGPT is serum glutamic pyruvic transaminase [also known as alanine aminotransferase (ALT)], an enzyme that is present in the same tissues as SGOT. Its appearance in serum is a marker of tissue damage similar to SGOT, but it is a more specific indicator of liver damage. The normal range of values for ALT (SGPT) is from 7 to 56 units per liter of serum.

Since CPK elevation of 5 fold or higher than normal serum levels provides that most reliable marker for muscle injury in rhabdomyolysis, it is taken as a marker of the disease and CPK elevation alone is regarded as within the scope of the present invention.

Complications:

Complications from rhabdomyolysis arise from the local effects of muscle cell lysis and the systemic effects of the substances released. When sarcolemmal integrity is compromised there are several ionic exchanges between the extracellular and intracellular compartments. These electrolyte and solute shifts may cause significant acute biochemical and hemodynamic abnormalities in the hours to days following muscle injury.

Shock:

The influx of fluid into the damaged muscle tissue may cause hypovolemia to the point of shock. Volume requirements soon after muscle injury may exceed 10 L/day, and two to three liters of saline per hour are often required during the initial management, followed by 300 to 500 ml/h once hemodynamic stability has been achieved. Failure to provide adequate volume replacement is probably the most frequent error made in the management of rhabdomyolysis. Indices of volume status such as urine output, urine sodium concentration and the blood urea nitrogen (BUN): creatine ratio may all be misleading, therefore assessment of volume status often needs central venous or pulmonary artery pressure monitoring, i.e., invasive hemodynamic monitoring. The insertion of a Swan-Ganz catheter provides a pulmonary capillary wedge pressure, which more accurately reflects fluid status.

Acute Renal Failure:

Probably the most significant complication of rhabdomyolysis is acute renal failure (ARF), seen in about 30% of patients. ARF may be caused by direct nephrotoxic effects of myoglobin, by its precipitation in renal tubules, or by its conversion to ferrihemate at a pH<5.6, which is both toxic to renal tubules and also precipitates. (see Holt et al. Pathogenesis and Treatment of Renal Dysfunction in Rhabdomyolysis. Intensive Care Medicine. Vol. 27: 803-811. 2001). In ARF secondary to rhabdomyolysis, hyperkalemia and hyperphosphatemia tend to occur early, and serum creatine concentration tends to be higher than expected for the level of azotemia (also called uremia, an excess of urea and other nitrogenous waste in the blood) owing to the release of previously formed creatine from damaged muscle. As a result of these imbalances dialysis may be required in 50-70% of patients. Particulary, emergency dialysis is indicated in uncontrolled hyperkalemia, acidosis, uremic encephalopathy or fluid overload. Serum myoglobin levels are not, however, reduced by hemodialysis.

Electrolyte Imbalances:

Hyperkalemia: The release of large amounts of potassium can cause life threatening hyperkalemia, which is typically less responsive to traditional therapies that rely on intracellular potassium shifting, such as the infusion of insulin and glucose, as the transport mechanisms that respond to this modality are likely to be impaired in injured muscle. Even if transported, potassium may leak from the intracellular compartment. If left untreated, hyperkalemia can lead to cardiac arrhythmias.

Hyperphosphatemia: This imbalance, caused by release of intracellular phosphate, may worsen hypocalcemia by decreasing the production of 1-25 dihydroxycholecalciferol. In the presence of normal calcium levels the calcium-phosphate product may increase and cause metastatic calcification. The release of purines and their subsequent hepatic conversion to uric acid may cause hyperuricemia, which, particularly in the setting of hypovolemia and low urine flow and pH, may cause sludging of urate crystals in the renal tubules, contributing to the pathogenesis of acute renal failure in rhabdomyolysis.

Anion gap acidosis: Sulfur-containing proteins released in large amounts can lead to hydrogen and sulfate loads that overwhelm renal excretory mechanisms, resulting in an anion gap acidosis, which may be severe. Anion gap is the difference between the sum of the measured cations and anions in the plasma or serum (based on sodium, potassium chloride and bicarbonate) and when less than or equal to 20 mmol/l, may indicate a bicarbonate-losing metabolic acidosis (since the kidneys regulate bicarbonate levels in the blood this may also be a sign of ARF). (Woodrow G, Brownjohn A M and Turney J H. The clinical and biochemical features of acute renal failure due to rhabdomyolysis. Renal Failure 1995; 17(4): 467-474).

Although systemic hypocalcemia predominates acutely in rhabdomyolysis, especially during low urine production in myoglobinuric renal failure, hypercalcemia may complicate the later diuretic phase during resolution of renal failure as calcium is mobilized from deposits in injured muscles by increased quantities of circulating 1-25 dihydroxycholecalciferol produced by the recovering kidneys.

Disseminated intravascular coagulation (DIC): may complicate rhabdomyolysis, and is most likely the result of activation of the clotting cascade by the intracellular components released from the lysed muscle cells. Overt clinical bleeding or thrombosis rarely complicates DIC in the case of rhabdomyolysis, and laboratory abnormalities allow a conclusive diagnosis that DIC is secondary to rhabdomyolysis. Because DIC leads to further ischemic damage, failure of serum CPK levels to decrease by approximately 50% every 48 may be an indicator of further ischemic muscle damage caused by DIC and appropriate treatment of this complication involves controlling or dissipating rhabdomyolysis by removing offending drug agent(s); running cultures for secondary infection and covering with antibiotics if needed, and replacing platelets if they are depleted below a critical level—usually below about 20,000.

Therapy:

Therapy of rhabdomyolysis is directed at two objectives: the first is the treatment of any reversible cause of muscle damage, as infections and compartmental ischemia; second is the management and prevention of complications. Because hypovolemia is often present, aggressive volume replacement is an urgent concern, as discussed above.

Electrolyte abnormalities in the acute stages of rhabdomyolysis often do require corrective intervention. Hyperkalemia should be corrected if potassium levels exceeds 6 mEq/L or cause conduction disturbances. Conventional therapy with insulin and glucose infusions, beta agonists and sodium bicarbonate may be ineffective because of loss of sarcolemmal (muscle cell membrane) integrity, and, therefore, early use of exchange resins and dialysis may be necessary. If hyperuricemia is severe (uric acid>20 mg/dl), allopurinol can be used. Hyperphosphatemia should be treated with phosphate binders. Calcium infusion can worsen the deposition in injured muscles and lead to higher levels of hypercalcemia in the diuretic phase of recovery from ARF. Therefore, calcium administration should only be used for the therapy of severe hyperkalemia or if ventricular dysfunction causes hypoperfusion.

Therapy aimed at preventing the onset of ARF is controversial. It is clear from animal studies that low urine volumes and aciduria potentiate the initial renal insult, with vigorous fluid administration to maximize urine flow and alkalinization with bicarbonate protecting against myoglobinuric renal injury.

Local therapy is extremely important in rhabdomyolysis of either traumatic or nontraumatic origin. Close attention should be paid to the decline of serum CPK levels. If does not fall by 50% over 48 h, a careful search should be made for evidence of increased tissue pressures in the involved muscle groups. If it is found, close attention should be focused on neurovascular function in affected limbs.

In the context of zonisamide therapy that results in rhabdomyolysis and/or serum CPK elevation, other complications must be treated as they arise; a skilled physician of emergency or internal medicine knows such treatments. For example, abruptly removing anti-epileptic drug therapy from an epileptic patient may result in more severe or more frequent seizures or even in status epilepticus. Therefore removal of zonisamide therapy may result in more severe seizures. However, a hospital physician or emergency medical technician will have access to other pharmacological interventions for short-term control of generalized seizure activity such as either intravenous lorazepam, at a dose of 0.1 mg/kg, or diazepam at 0.2 mg/kg. If sedatives prove insufficient, then a patient also may be administered fosphenyloin, or in status epilepticus, phenobarbital, with careful monitoring for respiratory depression. Intravenous administration is preferred since this route will provide the most rapid attainment of therapeutic serum levels. Given that seizures and status epilepticus are themselves causes of rhabdomyolysis, it is particularly important that such occurrences be avoided or minimized.

In some cases, it may be possible to reduce or taper-off the level of zonisamide to avoid elevated CPK, rhabdomyolysis, or other side-effects, while maintaining the therapeutic efficacy of the drug therapy. Such decisions may be made by an attending medical personnel, for example, after considering the severity of the side-effects in relation to the patient's need for continued zonisamide therapy. If the CPK elevation is not large enough to be concerning to the attending physician, they may consider cautiously maintaining zonisamide therapy or slowly taper administration of zonisamide and convert to an alternative AED.

Prevalence in Zonisamide Treated Patients:

The pharmacovigilance data that were collected, reviewed and analyzed provided the following information in respect of the incidence of rhabdomyolysis/CPK elevation in the zonisamide-treated patient population. To date, a total of 10 cases fulfill the criteria of potential rhabdomyolysis cases. These 10 cases were reviewed in detail for evaluation of possible safety signals. All 10 cases fulfill serious criteria. Of these 10 cases, seven (7) cases were reported as rhabdomyolysis and three (3) cases were reported as CPK increase.

For Adverse Events Reported as Rhabdomyolysis:

Of the seven (7) rhabdomyolysis cases, six (6) are verbatim cases from Dainippon and one (1) originates in the U.S. Of the seven (7) cases, three (3) were pediatric cases and four (4) were adult cases. Of the seven (7) cases, two (2) recovered, one (1) was recovering at time of report, two (2) had not recovered, and two (2) had a fatal outcome.

The development of rhabdomyolysis occurred between two (2) weeks and nine (9) years of the initiation of zonisamide treatment. Of the seven (7) rhabdomyolysis cases, two (2) cases have strong confounding factors, but the possibility of zonisamide involvement cannot be completely excluded. Two (2) cases have moderate confounding factors, and zonisamide involvement may be possible. Three (3) cases do not seem to have relevant confounding factors, and zonisamide involvement seems possible.

Based on these data, three (3) cases of rhabdomyolysis occurred during zonisamide treatment with no or only weak confounding factors present.

For Adverse Events Reported as CPK Serum Level Increase:

Of the three (3) cases of CPK increase, one (1) is a verbatim report from Dainippon, and two (2) originated from the U.S. Of the three (3) cases, two are pediatric patients and one is an adult. Of these three (3) cases, two (2) recovered and the outcome of the third is unknown. The development of CPK increase occurred between about two (2) days and six (6) weeks of the initiation of zonisamide treatment when documented.

Of the three (3) cases of CPK increase, one (1) case has strong confounding factors, but the possibility of zonisamide involvement cannot be completely excluded. One (1) case has weak confounding factors, and zonisamide involvement may be possible. One (1) case does not seem to have relevant confounding factors, and zonisamide involvement seem possible. Based on these data, two (2) cases of CPK increase occurred during zonisamide treatment with no or only weak confounding factors present.

Estimates:

Estimates of zonisamide exposure, based upon retail and mail order prescriptions, indicate that the number of unique patients taking zonisamide capsules in the U.S. is about 37,276 (total prescriptions per year/average number of prescriptions per patient per year less a calculated percentage decrease based on estimated annual dropouts) in the time between approval in 2000 and December 2002. Hospital patient data for that period, however, is not available and is not reflected in the estimates. Estimates of patient exposure for Japan indicate that the number of unique patients taking zonisamide is about 1,185,177 for time beginning with the approval in Japan through December 2002. Japanese data includes prescription and hospital patient data. Exposure from clinical trials are not included in the U.S. or Japanese exposure estimates. Based on these statistics, the estimated number of patients exposed to zonisamide in the U.S. and Japan is 1,222,453 unique patients. This is a rather conservative estimate, assuring that the number of patients actually exposed to zonisamide is unlikely to be higher than the estimate provided. Similarly, the incidences of rhabdomyolysis estimated herein are unlikely to be higher than calculated. Based on these data, two (2) cases of CPK increase occurred during zonisamide treatment with no or only weak confounding factors present. For the one (1) case reported in Japan, this amounts to an estimated incidence of 1:1,185,177 based upon estimated Japanese exposure. For the one (1) case reported in the US, this represents an estimated incidence of 1:37,276 based upon estimated US exposure. These two (2) cases of CPK increase represent a combined estimated incidence of 1:611,227 based upon the combined estimates of Japanese and US exposure. Thus, the overall estimated incidences of elevated CPK are 1:395,059 for Japanese cases, 1:18,638 for US cases and 1:244,491 for both Japanese and US cases, based on combining the reported cases of rhabdomyolysis and elevated CPK (the standard surrogate marker of muscle breakdown and typically seen in rhabdomyolysis).

Combining all the above cases of rhabdomyolysis and CPK increase, the estimated incidences of any patient who has experienced an elevated CPK (marker of muscle breakdown) are 1:400,000 for Japanese cases and 1:18,000 for US cases.

The following examples are provided to support the practice of the present invention and are not meant and should not be construed to limit the scope of the claims appended hereto.

### EXAMPLE 1

A ten-year old female experienced severe myalgia, increased CPK levels, and slight weakness of the proximal-leg muscles. The patient had a history of epilepsy and fetal alcohol syndrome. Zonisamide treatment was initiated on 20 Jul. 2002. On 27 Jul. 2002, the patient developed myalgia and slight weakness of the proximal leg muscles. The patient was hospitalized on 11 Sep. 2002 and the CPK serum level was found to be 962 U/I. Also on that same date zonisamide was discontinued. Subsequently the CPK levels decreased to 150 U/I which was in the normal range. The symptoms of myalgia and muscle weakness resolved on 13 Sep. 2002.

### EXAMPLE 2

A five-year old female patient who was receiving zonisamide for the treatment of breakthrough seizures developed myalgia and increased CPK levels. The reporting physician also indicated that the patient was using valproate alone, but the breakthrough seizures led to the addition of zonisamide as adjunctive therapy. Shortly after the initiation of zonisamide, the patient began to experience muscle cramps and myalgia which worsened over 3 to 4 weeks. The patient was hospitalized for myalgia and the CPK serum level was found to be about 900 U/I. After this finding, zonisamide was discontinued and CPK decreased to 304 U/I. The symptoms improved and the patient was discharged from the hospital before the symptoms had completely resolved. The reporting physician had scheduled a muscle specialist to rule out an autoimmune etiology of the adverse events, and reported the case as possibly related to zonisamide therapy.

While this invention has been described with respect to various specific examples and embodiments, it is to be understood that the invention is not limited thereby and should only be construed by interpretation of the scope of the appended claims.

1. A method of using zonisamide as an adjunctive therapy for partial seizures to improve the safety of such therapy comprising:
providing a patient with a therapeutically effective amount of zonisamide, and
informing the patient or the patient's guardian during the course of zonisamide therapy that muscle stiffness, muscle pain, muscle weakness, fever, discolored urine or altered consciousness are symptoms of rhabdomyolysis that require prompt medical evaluation if such symptoms are experienced by the patient.

2. The method of claim 1 wherein the therapeutically effective amount of zonisamide is from 25 mg to 600 mg.

3. The method of claim 1 wherein the therapeutically effective amount of zonisamide is provided in unit dose form.

4. The method of claim 1 wherein the therapeutically effective amount of zonisamide is provided in a unit dose form and in multiple doses to provide for a course of therapy.

5. The method of claim 4, wherein the unit dose is from 25 mg to 200 mg.

6. A method of using zonisamide as an adjunctive therapy for partial seizures to improve the health of a patient receiving such therapy comprising: providing a patient with a therapeutically effective amount of zonisamide, and
informing the patient or the patient's guardian during the course of such therapy that muscle stiffness, muscle pain, muscle weakness, fever, discolored urine or altered consciousness are symptoms of rhabdomyolysis that require prompt medical evaluation if such symptoms are experienced by the patient.

7. The method of claim 6 wherein the therapeutically effective amount of zonisamide is from 25 mg to 600 mg.

8. The method of claim 6 wherein the therapeutically effective amount of zonisamide is provided in unit dose form.

9. The method of claim 6 wherein the therapeutically effective amount of zonisamide is provided in a unit dose form and in multiple doses to provide for a course of therapy.

10. The method of claim 9, wherein the unit dose is from 25 mg to 200 mg.

11. A method of using zonisamide as an adjunctive therapy for partial seizures to reduce the risk of rhabdomyolysis in a patient receiving such therapy comprising:
providing the patient with a therapeutically effective amount of zonisamide, and
informing the patient or the patient's guardian during the course of zonisamide therapy that muscle stiffness, muscle pain, muscle weakness, fever, discolored urine or altered consciousness are symptoms of rhabdomyolysis that require prompt medical evaluation if such symptoms are experienced by the patient.

12. The method of claim 11 wherein the therapeutically effective amount of zonisamide is from 25 mg to 600 mg.

13. The method of claim 11 wherein the therapeutically effective amount of zonisamide is provided in unit dose form.

14. The method of claim 11 wherein the therapeutically effective amount of zonisamide is provided in a unit dose form and in multiple doses to provide for a course of therapy.

15. The method of claim 14, wherein the unit dose is from 25 mg to 200 mg.

16. A method of using zonisamide as an adjunctive therapy for partial seizures comprising:
enhancing the safety profile of zonisamide by informing a prescribing physician that creatine phosphokinase (CPK) elevation may result from zonisamide therapy
advising the physician to monitor a patient who is prescribed zonisamide as an adjunctive therapy for partial seizures for one or more symptoms chosen from the group of muscle stiffness, muscle pain, muscle weakness, fever, discolored urine and altered consciousness,
recommending that a laboratory serum measurement of the CPK level be performed and, if the level is elevated to about five times the normal level or higher, that the physician consider removing, reducing, or tapering off zonisamide dosing in the patient while initiating appropriate supportive therapy.

17. A method of using zonisamide as an adjunctive therapy for partial seizures comprising:
improving patient outcome by informing an emergency medical worker that a patient who is receiving zonisamide as an adjunctive therapy for partial seizures and exhibits muscle stiffness, muscle pain, muscle weakness, fever, discolored urine or altered consciousness, may be suffering from rhabdomyolysis; and
recommending performance of an appropriate diagnostic to determine if creatine phosphokinase (CPK) levels are about five times normal or higher,
and if CPK levels are about five times normal or higher, recommending that the worker initiate appropriate supportive therapy and discontinue or reduce zonisamide dosing in the patient.

18. The method of any of claim 17 wherein the diagnostic comprises measurement of serum creatine phosphokinase (CPK) or aldolase.

19. The method of claim 17 wherein the diagnostic comprises measurement of serum CPK-MM isoenzyme.

20. The method of any of claims 17 wherein the prescribed dosage of zonisamide is from 25 mg to 600 mg.

21. The method of claim 17 wherein the therapeutically effective amount of zonisamide is provided in unit dose form.

22. The method of claim 17 wherein the patient is receiving zonisamide in a therapeutically effective amount provided in a unit dose form and in multiple doses to provide for a course of therapy.

23. The method of claim 21 wherein the unit dose is from 25 mg to 200 mg.

24. A method of using zonisamide as an adjunctive therapy for partial seizures comprising:
providing packaging that includes a pharmaceutical formulation of zonisamide along with information providing a warning that zonisamide may cause rhabdomyolysis or creatine phosphokinase (CPK) elevation in some patients and that one or more symptoms chosen from the group of muscle stiffness, muscle pain, muscle weakness, fever, discolored urine and altered consciousness are potentially signs of rhabdomyolysis or CPK elevation; and
providing such packaging to a patient who has been prescribed zonisamide.

25. The method of claim 24 wherein the formulation contains a therapeutically effective amount of zonisamide of from 25 mg to 600 mg.

26. The method of claim 24 wherein the therapeutically effective amount of zonisamide is provided in unit dose form.

27. The method of claim 24 wherein the therapeutically effective amount of zonisamide is provided in unit dose form and in multiple doses to provide for a course of therapy.

28. The method of claim 24 wherein the unit dose is from 25 mg to 200 mg.

29. A method of using zonisamide as an adjunctive therapy for partial seizures comprising:
providing a patient with a therapeutically effective amount of zonisamide and a therapeutically effective amount of at least one other anti-epilepsy drug, and
informing the patient or the patient's guardian that muscle stiffness, muscle pain, muscle weakness, fever, discolored urine or altered consciousness are symptoms of rhabdomyolysis that require prompt medical evaluation if such symptoms are experienced by the patient.

30. The method of claim 29, wherein the patient or patient's guardian is informed by reference to a package drug insert.

31. A method of administering zonisamide as an adjunctive therapy for partial seizures comprising:
providing a patient with a therapeutically effective amount of zonisamide and a therapeutically effective amount of at least one other anti-epilepsy drug; and
informing the patient or the patient's guardian that muscle stiffness, muscle pain, muscle weakness, fever, discolored urine or altered consciousness are symptoms of rhabdomyolysis that require prompt medical evaluation if such symptoms are experienced by the patient.

32. The method of claim 31, wherein the patient or patient's guardian is informed by reference to a package drug insert.

33. A method of using zonisamide as an adjunctive therapy for partial seizures comprising:
informing the physician that creatine phosphokinase (CPK) elevation may result from zonisamide therapy
advising a physician prescribing zonisamide to a patient to monitor the patient for one or more symptoms chosen from the group of muscle stiffness, muscle pain, muscle weakness, fever, discolored urine and altered consciousness,
recommending that a laboratory serum measurement of the CPK level be performed and, if the level is elevated to about five times the normal level or higher, that the physician consider removing, tapering off, or reducing zonisamide dosing in the patient while initiating or maintaining other appropriate supportive therapy.

34. A method of using zonisamide as an adjunctive therapy for partial seizures comprising:
monitoring a patient who is receiving administrations of zonisamide for one or more symptoms chosen from the group of muscle stiffness, muscle pain, muscle weakness, fever, discolored urine and altered consciousness;
if one or more of said symptoms are observed, determining the serum CPK level of the patient; and
if the level of the patient's CPK is elevated to about five times the normal level or higher, reducing or tapering off the zonisamide dosing until the patient's CPK level is below five times the normal level.

35. The method of claim 34, wherein the zonisamide dosing is increased after the CPK level has dropped below five times the normal level.

36. A method of using zonisamide as an adjunctive therapy for partial seizures comprising:
monitoring a patient who is receiving administrations of zonisamide for one or more symptoms chosen from the group of muscle stiffness, muscle pain, muscle weakness, fever, discolored urine and altered consciousness;
if one or more of said symptoms are observed, determining the serum CPK level of the patient; and
if the level of the patient's CPK is elevated to about five times the normal level or higher, ceasing the zonisamide dosing until the patient's CPK level is below five times the normal level.

37. The method of claim 36, wherein the zonisamide dosing is restored after the CPK level has decreased below five times the normal level.
